(19) United States
(12) Patent Application Publication
(10) Pub. No.: US 2016/0206398 A1
(43) Pub. Date: Jul. 21, 2016

(54) MEDICAL IMAGING SYSTEM WITH MECHANICAL ARM

(71) Applicant: TELEFIELD MEDICAL IMAGING LIMITED, Hong Kong (CN)

(72) Inventors: Yongping Zheng, Hong Kong (HK); James Chung Wai Cheung, Hong Kong (HK); Tak Man Mak, Hong Kong (HK)

(21) Appl. No.: 14/915,272

(22) PCT Filed: Aug. 26, 2014
(86) PCT No.: PCT/CN2014/085196

§ 371 Date: Feb. 29, 2016

(30) Foreign Application Priority Data
Aug. 29, 2013 (CN) 201320532822.5

(51) Int. Cl. A61B 90/50; A61B 8/00

(52) U.S. Cl. CPC A61B 90/50; A61B 8/4218

(57) ABSTRACT

A medical imaging system with a mechanical arm (3) comprises a support (1), a probe (2), the mechanical arm (3), and a controller. The mechanical arm (3) is mounted on the support (1). The probe (2) is mounted at one end away from the support (1), of the mechanical arm (3). The controller can drive the mechanical arm (3) to drive the probe (2) to implement multi-degree-of-freedom movement. The medical imaging system uses a structure that the controller drives the mechanical arm (3) to drive the probe (2) to implement multi-degree-of-freedom movement, so that working intensity when staff such as doctors operates the probe can be lowered, thereby alleviating operation fatigue of the staff such as the doctors.

### TECHNICAL FIELD

The present application relates to a medical equipment field, in particular relates to a medical imaging system with a mechanical arm.

### BACKGROUND

At present, when conducting a three-dimensional imaging on a part of the patient by a medical imaging system, such part can be diagnosed and analyzed according to the imaging results. For example, an ultrasound imaging or an X-ray imaging can be used to diagnose the scoliosis of the patient, or other parts of the patient. When conducting a three-dimensional imaging on a part of the patient by an existing medical imaging system, the operator, such as a doctor and so on, should manipulate the probe to scan such part. However, in this way, the operation of the probe is rather inconvenient and easy to aggravate the fatigue of the operator, such as a doctor and so on.

### SUMMARY

An objective of the present application is to provide a medical imaging system with a mechanical arm capable of controlling a movement of a probe automatically, aiming at the technical problem that the probe operation of the existing medical imaging system is inconvenient.

According to one aspect, a medical imaging system with a mechanical arm is provided, including a support, a probe, the mechanical arm, and a controller. The mechanical arm is mounted on the support. The probe is mounted at one end away from the support, of the mechanical arm. The controller can control the mechanical arm to drive the probe for conducting a multi-degree-of-freedom movement.

In the medical imaging system with a mechanical arm according to the present application, the mechanical arm includes a fixing part mounted on the support, a motion arm in transmission connection with the fixing part, and a clamping mechanism in transmission connection with one end away from the fixing part, of the motion arm. The probe is mounted on the clamping mechanism.

In the medical imaging system with a mechanical arm according to the present application, the mechanical arm further includes a first transmission mechanism, via which the motion arm is in transmission connection with the fixing part. The first transmission mechanism further includes a first motor mounted on the fixing part, a second motor mounted on an output shaft of the first motor, a third motor mounted on an output shaft of the second motor. The output shaft of the first motor and the output shaft of the second motor are arranged mutually perpendicular to each other. The motion arm is mounted on an output shaft of the third motor.

In the medical imaging system with a mechanical arm according to the present application, the mechanical arm further includes a second transmission mechanism, via which the clamping mechanism is in transmission connection with one end away from the fixing part, of the motion arm. The second transmission mechanism further includes a fourth motor mounted on one end away from the fixing part, of the motion arm; and a fifth motor mounted on an output shaft of the fourth motor. The output shaft of the fourth motor and the output shaft of the fifth motor are arranged mutually perpendicular to each other. The clamping mechanism is mounted on an output shaft of the fifth motor.

In the medical imaging system with a mechanical arm according to the present application, the motion arm includes a first motion arm in transmission connection with the fixing part, a second motion arm in transmission connection with the first motion arm, and a third motion arm in transmission connection with the second motion arm, one end of the third motion arm away from the second motion arm is in transmission connection with the clamping mechanism.

In the medical imaging system with a mechanical arm according to the present application, the mechanical arm further includes a third transmission mechanism, via which the second motion arm is in transmission connection with the first motion arm. The third transmission mechanism further includes a sixth motor mounted on the first motion arm. The second motion arm is mounted on an output shaft of the sixth motor. The second motion arm and the output shaft of the sixth motor are arranged mutually perpendicular to each other.

In the medical imaging system with a mechanical arm according to the present application, the mechanical arm further includes a fourth transmission mechanism, via which the third motion arm is in transmission connection with the second motion arm. The fourth transmission mechanism further includes a seventh motor mounted on the second motion arm. The third motion arm is mounted on an output shaft of the seventh motor. The third motion arm and the output shaft of the seventh motor are arranged mutually perpendicular to each other.

In the medical imaging system with a mechanical arm according to the present application, the medical imaging system further includes a position sensor and a pressure sensor mounted on the mechanical arm, the controller can control a movement of the mechanical arm according to signals detected by the position sensor and the pressure sensor.

In the medical imaging system with a mechanical arm according to the present application, the clamping mechanism includes a connection element in transmission connection with the motion arm, a pair of clamping bodies extending from two opposite sides of the connection element and toward each other. The probe is clamped between the pair of clamping bodies.

In the medical imaging system with a mechanical arm according to the present application, the support has a vertical structure and the mechanical arm is arranged on one side of the support.

By implementing the medical imaging system with a mechanical arm according the present application, following benefits can be obtained. The medical imaging system can control the mechanical arm to drive the probe for conducting a multi-degree-of-freedom movement, thus reducing the work strength of operating the probe by the operator, such as a doctor and so on and alleviating their fatigue. Secondly, the medical imaging system employs a mechanical arm which can improve the repeatability and accuracy of measurement. Furthermore, the medical imaging system adopts the position sensor to collect the spatial position of the probe to control the movement route of the probe in real time with supports from the information provided by the collected images. In additional, the medical imaging system adopts the pressure sensor to monitor the acting force of the mechanical arm, thus effectively avoiding application of excessive force.

### BRIEF DESCRIPTION OF THE DRAWINGS

Hereinafter, embodiments of present invention will be described in detail with reference to the accompanying drawings, wherein:

FIG. 1 is a three-dimensional structure of the medical imaging system with a mechanical arm according to a first preferable embodiment of the present application.

FIG. 2 is an enlarged drawing of part A in FIG. 1.

FIG. 3 is a structural schematic diagram of the mechanical arm in FIG. 1 when it clamps with probe.

FIG. 4 is an exploded view of the mechanical arm shown in FIG. 1.

FIG. 5 is a three-dimensional structure of the medical imaging system with a mechanical arm according to a second preferable embodiment of the present application.

### DETAILED DESCRIPTION OF THE PREFERRED EMBODIMENT

These technical features, objects and effects of present application will be better understood from the following description and drawings.

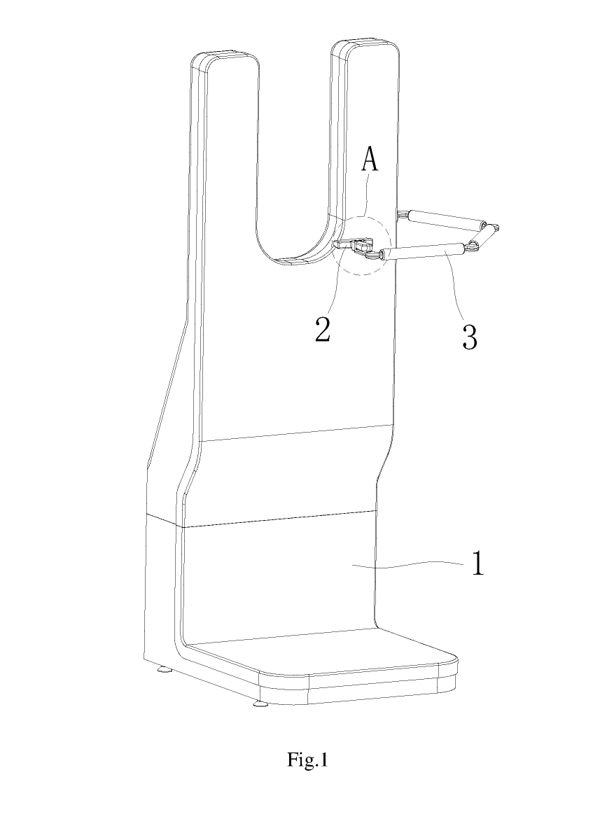

As shown in FIG. 1, the medical imaging system with a mechanical arm according to a first preferable embodiment of the present application includes a support 1, a probe 2, the mechanical arm 3, a controller (unshown), a position sensor (unshown), a pressure sensor (unshown).

As shown in FIG. 1, the support 1 has a vertical structure. The mechanical arm 3 is arranged on one side of the support 1. In the present embodiment, when the patient stands facing the support 1, the mechanical arm 3 is on the right side of the patient. The probe 2 is mounted on one end away from the support 1, of the mechanical arm 3. In the present embodiment, the probe 2 is an ultrasonic probe, which is used for imaging the spine of the patient. The installation height of the mechanical arm 3 is roughly matched with the spine position of the human body. In other embodiments of the present application, the probe 2 can also be used for imaging other parts of the patient.

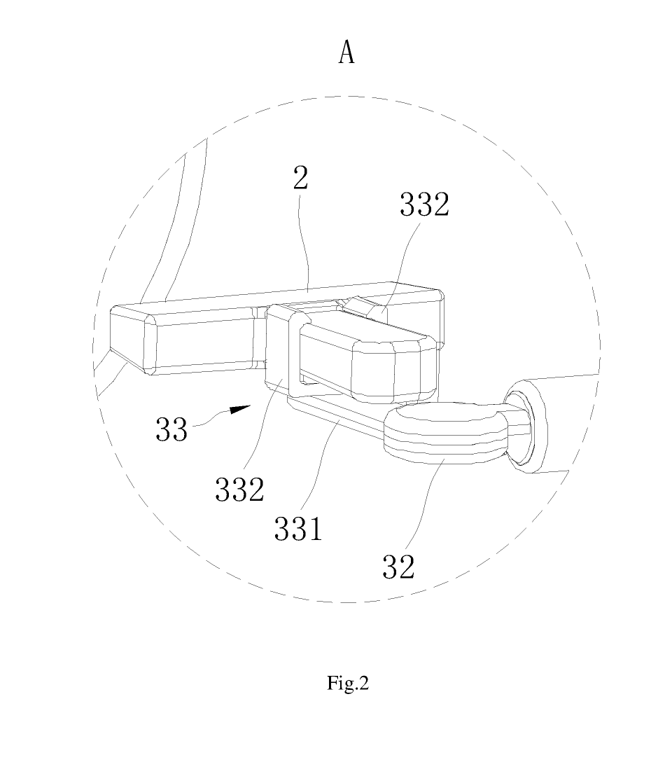

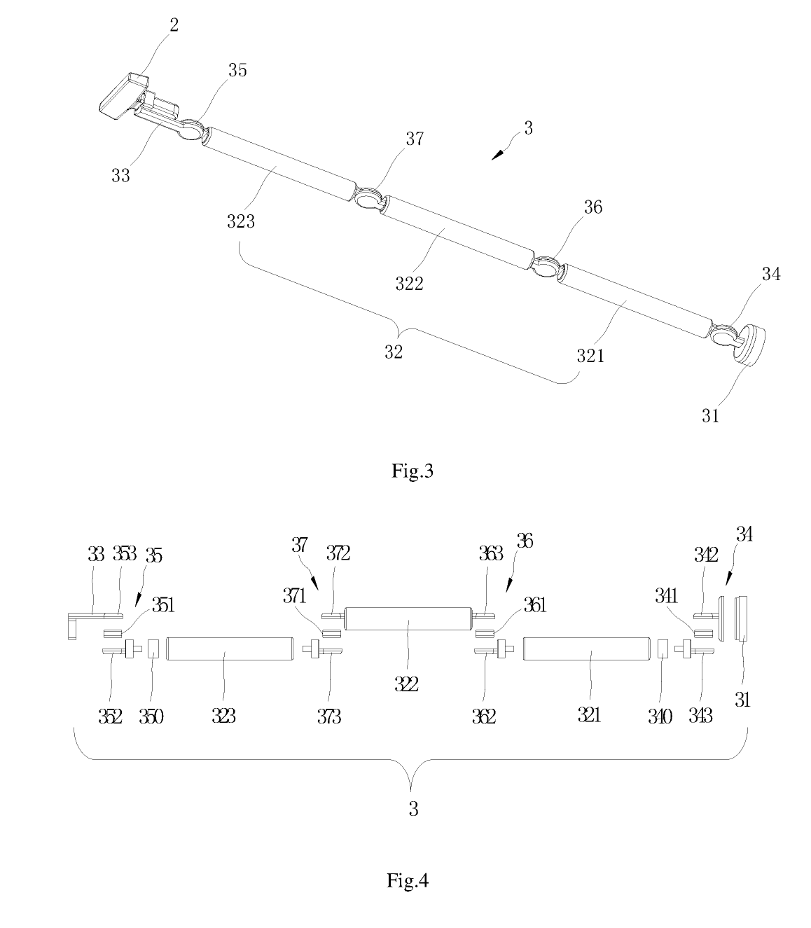

As shown in FIGS. 2-4, the mechanical arm 3 includes a fixing part 31, a motion arm 32, a clamping mechanism 33, a first transmission mechanism 34, a second transmission mechanism 35, a third transmission mechanism 36 and a fourth transmission mechanism 37. The fixing part 31 has a hollow cylinder structure, and is fixed on the side of the support 1. One end of the fixing part 31 far away from the support 1 is in transmission connection with the motion arm 32. The motion arm 32 includes a first motion arm 321, a second motion arm 322, and a third motion arm 323. The first motion arm 321 has a hollow rod structure, one end of which is in transmission connection with the fixing part 31, and the other end of which is in transmission connection with the second motion arm 322. The second motion arm 322 has a hollow rod structure, one end of which is in transmission connection with the first motion arm 321, and the other end of which is in transmission connection with the third motion arm 323. The third motion arm 323 has a hollow rod structure, one end of which is in transmission connection with the second motion arm 322, and the other end of which is in transmission connection with the clamping mechanism 33. The clamping mechanism 33 includes a connection element 331 and a clamping body 332. The connection element 331 has a bar structure, and is in transmission connection with the third motion arm 323. A pair of clamping bodies 332 extending from two opposite sides of the connection element 331 and toward each other is arranged. The probe 2 is clamped between the pair of clamping bodies 332.

As shown in FIG. 4, the first transmission mechanism 34 is used for realizing the transmission connection between the first motion arm 321 and the fixing part 31. The first transmission mechanism 34 includes a first motor (unshown), a second motor 341, a first gear set 342, a second gear set 343 and the third motor 340. The first motor is mounted inside the fixing part 31. The second motor 341 is in transmission connection with the output shaft of the first motor via the first gear set 342. The third motor 340 is in transmission connection with the output shaft (unshown) of the second motor 341 via the second gear set 343. The first motion arm 321 is mounted on the output shaft (unshown) of the third motor 340. In the present embodiment, the output shaft of the first motor and the output shaft of the second motor 341 are arranged mutually perpendicular to each other. The output shaft of the second motor 341 and the output shaft of the third motor 340 are arranged mutually perpendicular to each other.

As shown in FIG. 4, the second transmission mechanism 35 is used for realizing the transmission connection between the third motion arm 323 and the connection element 331. The second transmission mechanism 35 includes a fourth motor 350, a fifth motor 351, a third gear set 352 and a fourth gear set 353. The fourth motor 350 is mounted inside one end of the third motion arm 323. The fifth motor 351 is in transmission connection with the output shaft of the fourth motor 350 via the third gear set 352. The connection element 331 is in transmission connection with the output shaft (unshown) of the fifth motor 351 via the fourth gear set 353. In the present embodiment, the output shaft of the fourth motor 350 and the output shaft of the fifth motor 351 are arranged mutually perpendicular to each other.

As shown in FIG. 4, the third transmission mechanism 36 is used for realizing the transmission connection between the first motion arm 321 and the second motion arm 322. The third transmission mechanism 36 includes a sixth motor 361, a fifth gear set 362 and a sixth gear set 363. The sixth motor 361 is in transmission connection with the first motion arm 321 via the fifth gear set 362. The second motion arm 322 is in transmission connection with the output shaft (unshown) of the sixth motor 361 via the sixth gear set 363. In the present embodiment, the output shaft of the sixth motor 361 and the second motion arm 322 are arranged mutually perpendicular to each other.

As shown in FIG. 4, the fourth transmission mechanism 37 is used for realizing the transmission connection between the second motion arm 322 and the third motion arm 323. The fourth transmission mechanism 37 includes a seventh motor 371, a seventh gear set 371 and an eighth gear set 373. The seventh motor 371 is in transmission connection with the second motion arm 322 via the seventh gear set 371. The third motion arm 323 is in transmission connection with the output shaft (unshown) of the seventh motor 371 via the eighth gear set 373. In the present embodiment, the output shaft of the seventh motor 371 and the third motion arm 323 are arranged mutually perpendicular to each other.

In the present embodiment, the motion arm 32 is provided with three arms. In the other embodiments of the present application, there are other optional numbers of arms in the motion arm 32. In such a way, corresponding transmission mechanisms can be added between the two adjacent arms.

The position sensor and the pressure sensor are respectively mounted on the motion arm 32. Both of the position sensor and the pressure sensor are mounted on the second motor 341. The controller can control the operations of the first transmission mechanism 34, the second transmission mechanism 35, the third transmission mechanism 36 and the fourth transmission mechanism 37, respectively according to the signals detected by the position sensor and the pressure sensor, such as to enable the mechanical arm 3 to drive the probe 2 for conducting a multi-degree-of-freedom movement, thus reducing the work strength of operating the probe by the operator, such as a doctor and so on and alleviating their fatigue. In the present embodiment, the mechanical arm 3 can drive the probe 2 for conducting a six-degree-of-freedom movement. The position sensor can collect the spatial position of the probe 2, and the controller can control the movement route of the probe in real time based on the probe spatial position together with the information provided by the collected images. The pressure sensor can monitor the acting force of the mechanical arm, thus effectively avoiding application of excessive force. In the present embodiment, the scanning route of the probe 2 on the patient's spine can also be controlled according to a predefined movement route of the mechanical arm 3.

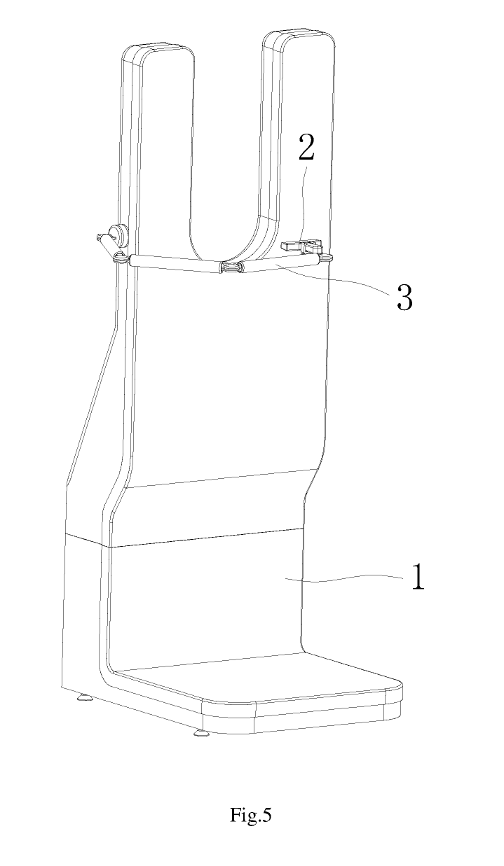

As shown in FIG. 5, the second preferable embodiment of the present application has provided a medical imaging system with a mechanical arm, which is different from the first preferable embodiment in that, the mechanical arm 3 is mounted on the position of the support 1. In the present embodiment, when the patient stands facing the support 1, the mechanical arm 3 is on the left side of the patient.

The present embodiment is described combining the attached drawings and the embodiments of the present application. Although the preferred embodiments of the invention have been described, one skilled in the art may make further changes and modifications to these embodiments as soon as he/she has learned the basic creative concepts. Therefore, the attached claims are intended to include the preferred embodiments and all changes and modifications falling into the scope of the present invention.

What is claimed is:

1. A medical imaging system with a mechanical arm including a support (1) and a probe (2), wherein, the medical imaging system further includes the mechanical arm (3), and a controller, wherein the mechanical arm (3) is mounted on the support (1), the probe (2) is mounted at one end away from the support (1), of the mechanical arm (3), wherein the controller can control the mechanical arm (3) to drive the probe (2) for implementing a multi-degree-of-freedom movement.

2. The medical imaging system with a mechanical arm according to claim 1, wherein the mechanical arm (3) includes a fixing part (31) mounted on the support (1), a motion arm (32) in transmission connection with the fixing part (31), and a clamping mechanism (33) in transmission connection with one end away from the fixing part (31), of the motion arm (32), wherein the probe (2) is mounted on the clamping mechanism (33).

3. The medical imaging system with a mechanical arm according to claim 2, wherein the mechanical arm (3) further includes a first transmission mechanism (34), via which the motion arm (32) is in transmission connection with the fixing part (31), the first transmission mechanism (34) further includes a first motor mounted on the fixing part (31), a second motor (341) mounted on an output shaft of the first motor, a third motor (340) mounted on an output shaft of the second motor (341), wherein the output shaft of the first motor and the output shaft of the second motor (341) are arranged mutually perpendicular to each other, the motion arm (32) is mounted on an output shaft of the third motor (340).

4. The medical imaging system with a mechanical arm according to claim 2, wherein the mechanical arm (3) further includes a second transmission mechanism (35), via which the clamping mechanism (33) is in transmission connection with one end away from the fixing part (31), of the motion arm (32); wherein the second transmission mechanism (35) further includes a fourth motor (350) mounted on one end away from the fixing part (31), of the motion arm (32) and a fifth motor (351) mounted on an output shaft of the fourth motor (350), the output shaft of the fourth motor (350) and the output shaft of the fifth motor (351) are arranged mutually perpendicular to each other, the clamping mechanism (33) is mounted on an output shaft of the fifth motor (351).

5. The medical imaging system with a mechanical arm according to claim 2, wherein the motion arm (32) includes a first motion arm (321) in transmission connection with the fixing part (31), a second motion arm (322) in transmission connection with the first motion arm (321), and a third motion arm (323) in transmission connection with the second motion arm (322), one end of the third motion arm (323) away from the second motion arm (322) is in transmission connection with the clamping mechanism (33).

6. The medical imaging system with a mechanical arm according to claim 5, wherein the mechanical arm (3) further includes a third transmission mechanism (36), via which the second motion arm (322) is in transmission connection with the first motion arm (321), the third transmission mechanism (36) further includes a sixth motor (361) mounted on the first motion arm (321), the second motion arm (322) is mounted on an output shaft of the sixth motor (361), the second motion arm (322) and the output shaft of the sixth motor (361) are arranged mutually perpendicular to each other.

7. The medical imaging system with a mechanical arm according to claim 5, wherein the mechanical arm (3) further includes a fourth transmission mechanism (37), via which the third motion arm (323) is in transmission connection with the second motion arm (322), the fourth transmission mechanism (37) further includes a seventh motor (371) mounted on the second motion arm (322), the third motion arm (323) is mounted on an output shaft of the seventh motor (371), the third motion arm (323) and the output shaft of the seventh motor (371) are arranged mutually perpendicular to each other.

8. The medical imaging system with a mechanical arm according to claim 1, wherein further including a position sensor and a pressure sensor mounted on the mechanical arm (3), wherein the controller can control a movement of the mechanical arm (3) according to signals detected by the position sensor and the pressure sensor.

9. The medical imaging system with a mechanical arm according to claim 2, wherein the clamping mechanism (33) includes a connection element (331) in transmission connection with the motion arm (32), and a pair of clamping bodies (332) extending from two opposite sides of the connection element (331) and toward each other, wherein the probe (2) is clamped between the pair of clamping bodies (332).

10. The medical imaging system with a mechanical arm according to claim 1, wherein the support (1) has a vertical structure and the mechanical arm (3) is arranged on one side of the support (1).
